(19) United States
(12) Patent Application Publication
(10) Pub. No.: US 2026/0140090 A1
(43) Pub. Date: May 21, 2026

(54) SOIL MONITORING

(71) Applicant: BAKER CONSULTANTS LIMITED, Matlock Derbyshire (GB)

(72) Inventors: Andrew BAKER, Matlock Derbyshire (GB); Carlos ABRAHAMS, Matlock Derbyshire (GB)

(21) Appl. No.: 19/118,868

(22) PCT Filed: Oct. 10, 2023
(86) PCT No.: PCT/EP2023/077971

§ 371 Date: Apr. 7, 2025

(30) Foreign Application Priority Data
Oct. 11, 2022 (GB) 2214980.1
Jan. 24, 2023 (GB) 2300992.1

(51) Int. Cl. G01N 29/12; G01N 29/24; G01N 29/44; G01N 33/24

(52) U.S. Cl. CPC G01N 29/12; G01N 29/24; G01N 29/4436; G01N 33/24; G01N 2291/02466

(57) ABSTRACT

Soil Monitoring Soil testing apparatus (101), for use in determining an indicator of soil health, comprises a probe (102) having a penetrating tip (107) for insertion in soil (100) to be tested, a sensor (106) operatively connected to the probe (102) and configured to provide an output indicative of sensed vibration, and a processing unit (103), operatively connected to the sensor (106), configured to acquire data indicative of vibration sensed in the soil (100) to be tested after the probe (102) has been located in a sensing position in the soil (100), the processing unit (103) configured to receive the output indicative of sensed vibration and store data derived therefrom for processing to determine an indicator of soil health based on the sensed vibration. A soil bioacoustic or ecoacoustic meter that comprises the soil testing apparatus (101). A method of determining an indicator of soil health using the soil testing apparatus (101).

### FIELD OF THE INVENTION

The present invention relates to soil monitoring, and more particularly to apparatus for and a method of determining an indicator of soil health.

### BACKGROUND OF THE INVENTION

Soil health refers to a soil's ability to function within an ecosystem to sustain plants, animals, and humans. Different soil characteristics may be desirable in different environments and/or for different uses, and various ways to test or analyse a soil are known. Soil assessment may involve determining such properties as, for example, pH level, texture, organic matter content, moisture content, nutrient balance, electrical conductivity.

A relatively new field and developing field is that of soil bioacoustics, wherein the science of bioacoustics, which is tried-and-tested in air, is used to measure the biological activity of organisms that generate noise or vibrations. How “noisy” the soil is an indicator of how “alive” the soil is. Soil bioacoustics is based on the same general principle as acoustic ecology (ecoacoustics), in which the production, transmission and reception of sounds in a natural environment, such as from birds, bats, frogs, insects and other living things that make noise, is studied and the acquired data, including sound recordings, used to measure the health of the natural environment. In soil, a greater level of sound is indicative of a healthier soil, comprising more organic matter, a better structure and beneficial fauna.

It is desirable to provide a soil testing device that is usable, in the field, to acquire data indicative of sensed vibration in the soil, detected from biological activity, from which an indicator of soil health can be determined.

### SUMMARY OF THE INVENTION

Apparatus for measuring sound/vibrations in soil is provided. The apparatus generally comprises: means for detecting sound/vibrations in soil, means for deriving data from the detected soil sound/vibrations, and means for processing the derived data. An indicator of soil health based on the detected soil sound/vibrations can be determined. From monitoring the indicator, changes in soil “noisiness”, which may for example indicate an increasing or decreasing trend or a sudden crash or boom in sound/vibrations, can be identified.

A method of determining an indicator of soil health using apparatus for measuring sound/vibrations in soil is provided.

A soil bioacoustic meter, or soil ecoacoustic meter, is provided.

According to a first aspect there is provided soil testing apparatus for use in determining an indicator of soil health as clamed in claim 1. A probe has a penetrating tip for insertion into soil to be tested, to locate the probe in a sensing position in the soil in which the probe is in resting contact with the soil. A sensor, operatively connected to the probe, is functional to detect vibration in the soil to be tested when the probe is in the sensing position in the soil to be tested, the sensor configured to provide an output indicative of sensed vibration. A processing unit, operatively connected to the sensor, is functional to acquire data indicative of vibration sensed in the soil to be tested when the probe is in the sensing position in the soil, the processing unit configured to receive the output indicative of sensed vibration from the sensor and store data derived therefrom for processing to determine an indicator of soil health based on sensed vibration in the soil to be tested during a period of sensing in which the probe is in the sensing position in the soil.

Data derived from the sensor output may be processed on-board. An indicator of soil health based on sensed vibration may be determined by on-board processing. The determined indicator of soil health may be output for display on a display unit of the processing unit of the soil testing apparatus. Alternatively, or additionally, data derived from the sensor output may be stored on-board for transfer to another device for processing. The determined indicator of soil health may be output for display on a display unit of a remote device.

The sensor may comprise a contact microphone or an accelerometer.

The sensor may be comprised by a sensor unit that comprises at least one other sensor. The or each other sensor may be any suitable type, which may be the same as or different from the sensor.

The probe may be a rigid probe. This feature serves to assist the insertion of the probe into soil to be tested, for example when the soil is significantly compacted and so more resistant to penetration or when the soil contains hard material, for example stones or foreign material, that can impede the advancement of the probe into the soil. The feature also serves to enable a user to visually gauge the position/depth of the probe beneath the surface of the soil when in use.

The processing unit may be operatively connected to the sensor by a wired connection or a wireless connection. The processing unit may be selectively operatively connected to the sensor by a wired or a wireless connection.

The soil testing apparatus may be provided as a manually portable apparatus. This enables a user to conveniently transport the soil testing apparatus to and between different sites at which soil is to be tested.

The processing unit may comprise an enclosure. The enclosure may house/support componentry of the processing unit. The enclosure may be a waterproof housing. This feature serves to protect componentry housed within the enclosure, for example when the soil testing apparatus is in use in the field.

The processing unit and probe may be connected to form a single device. The sensor may be packaged with the processing unit. The sensor packaged with the processing unit may be housed by an enclosure of the processing unit that houses/supports componentry of the processing unit.

The processing unit and probe may be formed as separated devices that are connected by a wired or a wireless connection. The sensor may be packaged with the probe.

The processing unit may comprise a microprocessor. The processing unit may comprise an input device arrangement for conveying an input to the microprocessor. The processing unit may comprise an output device arrangement for conveying an output from the microprocessor.

The processing unit may comprise a data storage device. The data storage device may comprise non-volatile memory. The data storage device may be in communication with an expandable memory card slot.

The processing unit may comprise a communication port. The communication port may allow data transfer or both data transfer and power transfer.

The processing unit may comprise a battery power source. The battery power source may be a removable battery power source. The battery power source may be a rechargeable battery power source. The processing unit may comprise a power on/off element.

The processing unit may comprise at least a microprocessor, a data storage device, a communication port, a battery power source, and a power on/off element.

The processing unit may comprise a display unit. The display unit may comprise one or more screens. The display unit may be used to convey information about general device operation and/or readings.

The processing unit may comprise an audio output device. The audio output device may be configured to output any suitable sound or sounds. The audio output device may be used to convey information about general device operation and/or readings.

The processing unit may comprise an illumination device. The processing unit may comprise more than one illumination device. The or each illumination device may be configured to emit light at any suitable colours or colours, at any suitable brightness level or levels, and in accordance with any suitable pattern or patterns. The illumination device or devices may be used to convey information about general device operation and/or readings.

The processing unit may comprise a reading routine activation element for initiating the taking and recording of sensed vibration readings. Sampling may be performed any suitable period and at any suitable sampling rate. Recording may begin after a short delay from the reading routine activation element being activated.

The processing unit may comprise a timer device for tracking a period during which readings of sensed vibration are taken and recorded. The tracked period may be a pre-determined period. The timer device may be a countdown timer.

The processing unit may comprise a wireless communication module. The wireless communication module may be configured to allow communication with a global navigation satellite system. Alternatively, or additionally, the wireless communication module may be configured to allow communication with a remote device.

The processing unit may comprise at least a microprocessor, a data storage device, a communication port, a battery power source, and a power on/off element. The processing unit may further comprise at least one of a display unit, an audio output device, an illumination device, a reading routine activation element for initiating the taking and recording of sensed vibration readings, a timer device for tracking a period during which readings of sensed vibration are taken and recorded, and a wireless communication module.

The soil testing apparatus may be configured to record the frequency and amplitude of sensed vibration.

The processing unit may be configured to perform analysis of data derived from the output indicative of sensed vibration to generate an indicator of soil health based on sensed vibration. The generated indicator of soil health based on sensed vibration takes the form of a number on a proprietary scale. The proprietary scale may comprise a numerical scale with the generated indicator being a value N on the numerical scale. The numerical range of the proprietary scale may extend from a lower numerical value of zero to an upper numerical value of X, with a lower value indicating a lesser extent of detected vibration/sound (soil “noisiness”) and a higher value indicating a greater extent of detected vibration/sound (soil “noisiness”). The proprietary scale may be linear or non-linear.

The analysis may comprise processing the data derived from the output indicative of sensed vibration with reference to pre-obtained data indicative of vibration sensed in soil. The pre-obtained data may be derived from prior use of the soil testing apparatus.

Repeated use of the soil testing apparatus at a particular site, to monitor soil health at that site based on detected soil sound/vibrations, enables an increase or a decrease in soil noisiness, and hence a change in how “alive” the soil is, to be identified.

Data acquired from use of the soil testing apparatus to test the soil health at a site having one or more known characteristics can be used to create a reference pattern/signature/profile that may be usable to obtain an initial impression of whether the soil health at another site sharing the one or more known characteristics is similar or dissimilar.

According to a second aspect there is provided a soil bioacoustic or ecoacoustic meter comprising the soil testing apparatus of the first aspect, as claimed in claim 24.

According to a third aspect there is provided a method of determining an indicator of soil health, comprising: using the soil testing apparatus of the first aspect to acquire data indicative of vibration sensed when the probe is inserted into soil to be tested, and processing the acquired data to determine an indicator of soil health based on sensed vibration, as claimed in claim 25.

A soil testing apparatus, for use in determining an indicator of soil health, comprising a probe having a penetrating tip for insertion in soil to be tested, a sensor operatively connected to the probe and configured to provide an output indicative of sensed vibration, and a processing unit, operatively connected to the sensor, configured to acquire data indicative of vibration sensed in the soil to be tested after the probe has been located in a sensing position in the soil, the processing unit configured to receive the output indicative of sensed vibration and store data derived therefrom for processing to determine an indicator of soil health based on the sensed vibration, is disclosed. A soil bioacoustic or ecoacoustic meter that comprises the soil testing apparatus is further disclosed. A method of determining an indicator of soil health using the soil testing apparatus is also disclosed.

The present invention enables the detection, assessment, evaluation and monitoring of sounds in soil, such as those arising from worms and other invertebrates, which can provide an insight into the health of the soil. The present invention enables quantification of soil health based on vibration sensed in the soil.

Noise generated by biological activity within the soil can be recorded and used to gauge the health of the soil. This, in turn, can be used in a wider assessment of the natural environment. For example, some birds, such as blackbirds, have acute hearing that allows them to detect worms and insects that are moving within the ground, out of sight; an environmental disturbance that significantly reduces the worm population within a particular area could decrease the attractiveness of that locality to certain birds, which could be noticeable in the results of regular wildlife surveying of that area.

### DRAWINGS

The present invention will now be more particularly described, with reference to the accompanying drawings, in which:

FIG. 1 shows apparatus according to an example of the present invention;

FIG. 2 shows an alternative arrangement of the apparatus of FIG. 1;

FIG. 3 shows componentry of the apparatus, in accordance with the example of FIG. 1;

FIG. 4 shows features of the apparatus of the example of FIG. 1;

FIG. 5 shows an example presentation of an indicator of soil health derived from data obtained from use of the apparatus of the example of FIG. 1 or of FIG. 2; and

FIG. 6 shows steps in a method according to the present invention.

### DESCRIPTION

The present invention will now be more particularly described, with reference to the accompanying drawings (FIG. 1 to 6).

Illustrative embodiments and examples are described below in sufficient detail to enable those of ordinary skill in the art to embody and implement the apparatus described herein. It is to be understood that embodiments and examples can be provided in many alternate forms falling with the scope of the invention.

Unless otherwise defined, all terms (including technical and scientific terms) used herein are to be interpreted as is customary in the art. In addition, features referred to herein in the singular can number one or more, unless the context clearly indicates otherwise. Similarly, the terms “comprises”, “comprising”, “includes”, “including”, “has” and/or “having” when used herein, specify the presence of the stated feature or features, and do not preclude the presence or addition of one or more other features, unless the context clearly indicates otherwise.

In the following description, all orientational terms, such as upper, lower, radially, and axially, are used in relation to the drawings and should not be interpreted as limiting on the invention, unless the context clearly indicates otherwise.

The drawings are not necessarily drawn to scale, and in some instances the drawings may have been exaggerated or simplified for illustrative purposes only.

Apparatus for, and a method for, use in soil monitoring is provided, more particularly for determining an indicator of soil health that is based on detected noise/vibration.

Soil testing apparatus for use in determining an indicator of soil health is disclosed that comprises a probe, a sensor operatively connected to the probe and configured to provide an output indicative of sensed vibration, and a processing unit, operatively connected to the sensor, for acquiring data indicative of vibration sensed when the probe is inserted into soil to be tested, the processing unit configured to receive the output indicative of sensed vibration and store data derived therefrom for processing to determine an indicator of soil health based on sensed vibration. A soil bioacoustic or ecoacoustic meter comprising the soil testing apparatus is also disclosed. A method of determining an indicator of soil health, comprising using the soil testing apparatus is further disclosed.

Apparatus 101 according to an example of the present invention is shown in FIG. 1. The apparatus 101 comprises a probe 102, for insertion into soil 100, as illustrated, and a processing unit 103. The processing unit 103 comprises an enclosure 104 that houses/supports componentry, which is indicated generally at 105. The apparatus 101 further comprises a sensor 106, in this example a contact microphone or accelerometer, operatively connected to the probe 102.

The probe 102 has a penetrating tip 107 that is insertable into the soil 100 to locate the probe 102 in a sensing position within the soil 100, such as the sensing position illustrated in FIG. 1. The penetrating tip 107 of the probe 102 may have any suitable shape for facilitating insertion into soil to be tested, for example comprising a conical profile. In an example, the probe 102, and penetrating tip 107 thereof, are designed to allow the probe 102 to be manually driven into soil to be tested, and, in particular, soil in situ in the field (in other words, the probe 107 is designed to be manually pushed into the ground). The probe 102 may be provided with indicia indicating an extent that the penetrating tip 107 should be advanced into the soil to be tested to locate the probe in a sensing position within the soil. For example, a depth, represented by a distance along the probe 102 from the penetrating tip 107 may be marked on the probe 107. In an example, a depth range, between “minimum” and “maximum” distances marked along the probe 102 from the penetrating tip 107, may be shown.

It is to be appreciated that, for example, different wildlife populations, for example worm populations, may be anticipated to be present at different depths beneath the surface. Therefore, probe depth may be a useful factor in distinguishing sounds from different wildlife populations in the soil.

When in a sensing position in soil to be tested, the probe 102 is in resting contact with the soil. In other words, the probe 102 is stationary in the soil. An important aspect of the present invention is that the vibration sensing using the apparatus 101 is performed after the probe 102 has been driven into the soil to be tested and is no longer in motion. The apparatus 101 of the present invention thus differs from prior art devices for assessing soil structure (in particular, for determining soil particle size) that are designed to detect sound as a probe is being moved into the soil.

Another important aspect of the present invention is that the probe 102 is minimally invasive. As the penetrating tip 107 advances into the soil to be tested, the probe 102 displaces the soil it touches. In a preferred example, the probe 102 is designed to leave as little a trace as possible of having been accommodated in the body of the soil after its withdrawal from the soil. The apparatus 101 of the present invention thus differs from prior art devices for use in assessing soil that are designed to remove a core of soil for testing in a laboratory, and from prior art devices for assessing (and monitoring) soil that are designed to be placed (and left) within a pre-formed pit within the soil.

In a preferred embodiment, the apparatus 101 is provided as a manually portable apparatus.

According to the example shown in FIG. 1, the probe 102 and processing unit 103 of apparatus 101 are connected to form a single device. According to the illustrated arrangement, the contact microphone or accelerometer 106 is packaged with the processing unit 103.

The housing 104 is fabricated and constructed to possess properties that render it suitable for outdoor use, for example in respect of weatherproofing and robustness. In a preferred embodiment, the housing 104 is waterproof. The housing 104 may have any suitable dimensions.

The probe 102 may be any suitable type and may have any suitable dimensions. In an embodiment, the probe 102 is a rigid probe.

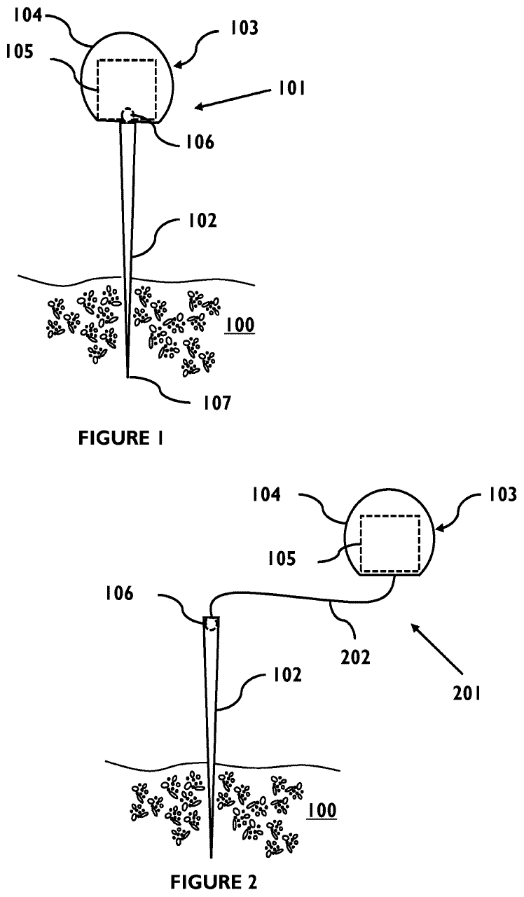

Apparatus 201 according to an alternative example of the present invention is shown in FIG. 2. According to the illustrated example, the probe 102 and processing unit 103 of apparatus 201 are provided as separate devices that are operatively connected by a wired or wireless communication connection 202. In this illustrated example the communication connection is a wired communication provided by a connection wire. According to the illustrated arrangement, the contact microphone or accelerometer 106 is packaged with the probe 102.

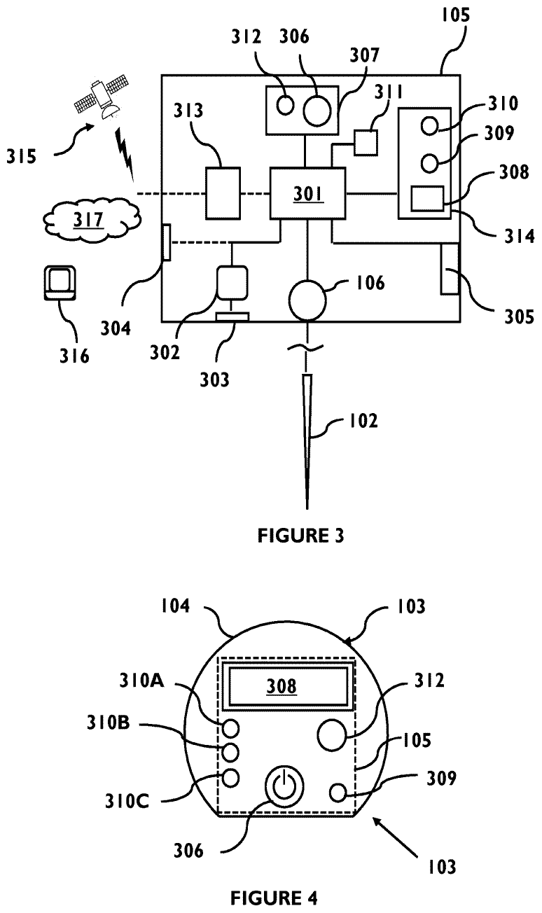

Reference will now be made to FIGS. 3 and 4, which show features of the apparatus 101 (soil bioacoustic meter or soil ecoacoustic meter) of the example of FIG. 1 in further detail.

In a specific example, the processing unit 103 comprises at least:
the contact microphone or accelerometer 106 (to which the probe 102 is attached);
a microprocessor 301;
a data storage device 302, optionally in communication with an expandable memory card slot 303;
a communication port 304, in communication with the data storage device 302;
a battery power source 305;
a power on/off element 306.

The processing unit may comprise an input device arrangement for conveying an input to the microprocessor. The processing unit may comprise an output device arrangement for conveying an output from the microprocessor.

The power on/off element 306 may be part of an input device arrangement, indicated generally at 307, which may comprise other components for or associated with conveying an input to the microprocessor 301.

In an embodiment, the data storage device 302 comprises non-volatile memory.

The communication port 304 may allow data transfer or both data and power transfer. In an embodiment, the communication port 304 comprises a USB port.

The battery power source 305 may comprise at least one removable, non-rechargeable battery or at least one rechargeable battery. In an embodiment, a rechargeable battery is provided that is rechargeable via a power transfer cable connectable to the processing unit, for example the communication port 304 or a separate recharging port (not illustrated).

The power on/off element 306 may be any suitable type, for example, a button, a rocker switch, a slide switch, a dial.

In an example, the processing unit 103 may further comprise one or more of the following components:
a display unit 308;
an audio output device 309;
an illumination device 310;
a timer device 311;
a reading routine activation element 312;
a wireless communication module 313.

Display unit 308 and/or audio output device 309 and/or illumination device 310 may be part of an output device arrangement, indicated generally at 314, which may comprise other components which may comprise other components for or associated with conveying an output from the microprocessor 301.

The display unit 308 may be any suitable type comprising one or more screens. In an embodiment, the display unit 308 is an LED screen. The display unit 308 may be used to convey information about general device operation and/or readings.

The audio output device 309 may be configured to output any suitable sound or sounds, which may, for example, be or comprise beeps. The audio output device 310 may be used to convey information about general device operation and/or readings. Sound may be used, for example, to indicate when readings are/are not being taken. For example, a beep may be output to indicate the start and the end of a period during which readings are being taken and/or a regular beep may be output while readings are being taken.

The illumination device 310 may be any suitable type. The illumination device 310 may be used to convey information about general device operation and/or readings. In an example the illumination device 310 is an LED light. The illumination device 310 may be configured to emit light at any suitable one or more colour and at any suitable one or more brightness levels and in accordance with any one or more suitable patterns (for example, continuous or flashing). More than one illumination device 310 may be provided, for example illumination devices 310A, 310B and 310C of FIG. 4. A plurality of illumination devices 310 may be used in different ways to convey information about general device operation and/or readings. Illumination may be used, for example, to indicate whether readings that have been taken indicate a soil “noisiness” above or below a predetermined threshold or outside a predetermined range of a soil “noisiness” determined previously for the soil being tested.

The timer device 311 may be any suitable device. In an embodiment, the timer device 311 comprises a countdown timer. The timer device 311 is usable to track a period, which in a preferred embodiment is a pre-determined period, during which readings are taken and recorded.

The reading routine activation element 312 may be any suitable type, for example, a button. The reading routine activation element 312 is usable to initiate the taking of readings. In an embodiment, operation of the reading routine activation element 312 triggers a short delay before recording begins. In a preferred embodiment, operation of the reading routine activation element 312 triggers a short delay before recording begins for a pre-determined period. In an example, sampling is performed over a period in the range of 15 s to 60 s, although it is to be appreciated that recording over any suitable time and at any suitable sampling rate may be carried out.

The wireless communication module 313 may allow communication with a global navigation satellite system, for example the Global Positioning System (GPS), as indicated at 315. This is usable to associate readings taken with a location.

The wireless communication module 314 may, additionally or alternatively, allow communication with a remote device, such as computer 316. Communication may be direct or indirect, and using any suitable network and protocol. For example, communication may be via a cellular network, which may have capabilities meeting a 3G, 4G or 5G standard as defined by the International Telecommunications Union (ITU), via a local area network (WLAN), for example Bluetooth or Wi-Fi, or via a wide area network, for example the internet, indicated generally at 317.

Soil noise/vibration readings taken using the apparatus 101 can be stored locally and processed on-board and/or stored for subsequent transfer to another device for processing. Data, in raw and/or processed form, may be transferrable from the apparatus 101 to another device for longer-term storage so that it is available for future analysis, which may involve other indicators of soil health.

In an embodiment, the apparatus is usable to record the frequency and amplitude of detected noise/vibration, and the frequency and/or the amplitude of the recording may be analysed for providing an indicator of soil health based on the detected sound/vibration.

In an embodiment, the frequency and the amplitude of recorded soil sound/vibration is analysed to produce an indicator of soil health that takes the form of a number on a scale.

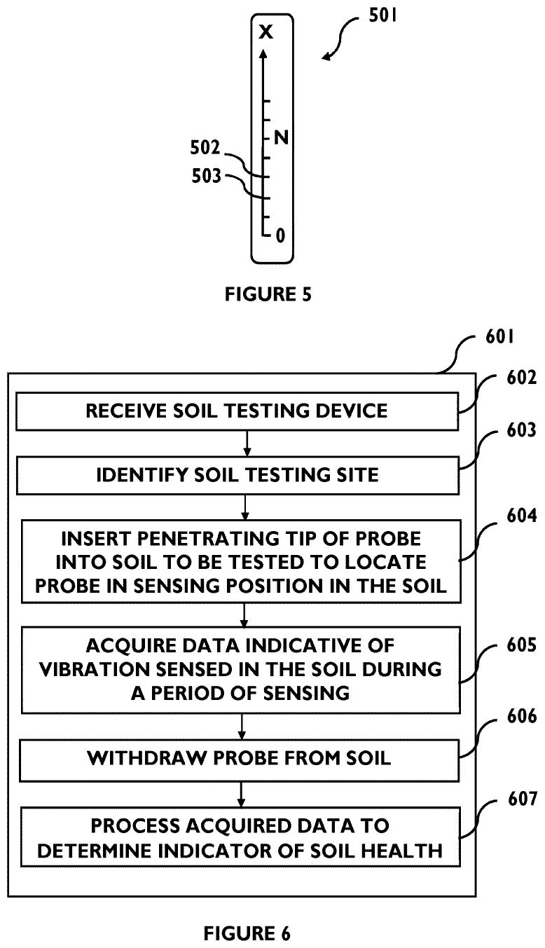

An example proprietary scale 501 is illustrated in FIG. 5, on which a value N produced from analysing a soil sound/vibration recording is presented. The numerical range of the illustrated scale 501 is from a lower numerical value of zero and an upper numerical value of X, with a lower value indicating a lesser extent of detected noise/vibration and a higher value indicating a greater extent of detected noise/vibration. The upper numerical value of X may be any suitable number. In this illustrated example, intervals, such as intervals 502 and 503 are indicated. A linear or a non-linear scale may be utilised.

It is to be understood that in some applications only the value N is output (as is the case with many types of known meter). For example, if the apparatus 101 of FIG. 1 has on-board processing capability, the value N can be displayed by the display unit 308, otherwise it could be displayed on a screen of or operatively connected to a remote device, such as computer 316.

In an example, a weighting factor may be applied to the scale 501, depending on the environment of the soil being tested. Apparatus calibration and/or data processing to generate a value N could be undertaken according to different requirements relating to for, for example, accuracy, precision, resolution, sensitivity.

Advantageously, the probe can be inserted into the soil to be tested and the reading routine activated to initiate the taking of readings during a pre-determined duration. Repeating the testing allows for trends or sudden changes to be identified. For example, regular testing of soil noise/vibration can be used to determine whether a soil management program is effective in increasing bioactivity or to confirm that an environmental event has had a negative effect on soil vitality.

By way of example, a method of using the soil testing apparatus for determining an indicator of soil health may comprise the steps of:
(i) using the soil testing apparatus to acquire first data for soil at a place A at time TI;
(ii) using the soil testing apparatus to acquire second data for soil at the place A at a later time T2; and
(iii) comparing the second data acquired at step (ii) with the first data acquired at step (i) to identify whether the recorded sound/vibration of the soil is stable, increasing or decreasing.

It is to be appreciated that soil sound/vibration readings may be recorded using any suitable format, for example an existing audio file format.

An example method 601 of use of a soil testing device according to the present invention, such as a soil bioacoustic or ecoacoustic meter comprising the soil testing apparatus 101 or 201 of the present invention, will now be described with reference to FIG. 6.

At step 602, the soil testing device is received. Any preparatory actions, which may comprise device function/resource checking (for example, in relation to battery power and/or available memory) can be carried out.

At step 603, a soil testing site is identified. The soil testing site may be at any suitable geographical location and in any suitable terrain, and could, for example, be in an agricultural field, in woodland or a clearing, grassland, marshland, or other natural habitat.

It is to be appreciated that step 603 may be performed before or at the same time as step 602.

At step 604, the penetrating tip of the probe of the soil testing device is inserted into the soil to be tested to locate the probe in a sensing position in the soil.

At step 605, data indicative of vibration sensed in the soil during a period of sensing is acquired. At this step, vibration sensing is performed using the soil testing device while the probe remains in the sensing position in the soil. The soil testing device may be activated to initiate a reading routine to take and record sensed vibration readings over a sensing period or over multiple sensing periods. The vibrations sensed correspond to biological sounds within the soil arising from the activity of organisms within the soil, for example insects moving, eggs hatching, small mammals burrowing, creatures communicating, or roots growing. The sounds captured during sensing can be interpreted in the context of how “alive” the soil, and hence how healthy the soil is at the time of testing.

At step 606, the probe is withdrawn from the soil. The probe may be cleaned as appropriate.

At step 607, data acquired from vibration sensing carried out at step 605 is processed to determine an indicator of soil health.

It is to be appreciated that step 607 may be performed before or during step 606, depending on when any on-board processing functionality is used or if the data processing is performed solely off-board.

Apparatus for measuring sound/vibrations in soil is thus provided, the apparatus generally comprising means for detecting sound/vibrations in soil, means for deriving data from the detected soil sound/vibrations, and means for processing the derived data to provide an indicator of soil health based on the detected soil sound/vibrations. A soil bioacoustic meter or soil ecoacoustic meter is thus provided.

A method of determining an indicator of soil health using the apparatus is also provided.

From monitoring the indicator, changes in soil “noisiness”, which may for example indicate an increasing or decreasing trend or a sudden crash or boom in sound/vibrations, can be identified. Soil noise/vibration recordings may be analysed with the objective of identifying patterns/signatures/profiles that could be useful in soil health assessment. Features in readings may be detectable with reference to, for example, climate/sub-climate, underlying geology or how cultivated a site is. For example, differences in noise readings taken from soil of different types (sandy, clay etc.) and/or with different surfaces (bare, grass-covered etc.) may be discernible, as may, by way of a further example, differences in sounds recorded in soil at different distances from a body of water (river, lake etc.)

The apparatus of the present invention is convenient to use and is designed so that the quality of the data obtained is not dependent on user interaction. Data obtained using the present invention can assist in, for example, guiding agricultural practices to improve soil management and limit undesirable soil degradation, to support the natural world and, importantly, food supplies across the globe.

Although illustrative embodiments and examples of the invention have been disclosed in detail herein, with reference to the accompanying drawings, it is to be understood that the invention is not limited to the precise embodiment and examples shown and/or described and that various changes and modifications can be effected therein by one skilled in the art without departing from the scope of the invention as defined by the appended claims.

1. A soil bioacoustic or ecoacoustic meter soil testing apparatus for use in determining an indicator of soil health of a site based on biological activity of organisms that generate noise or vibrations, the soil testing apparatus, comprising:
a probe having a penetrating tip for insertion into soil to be tested, to locate the probe in a sensing position in the soil in which the probe is in resting contact with the soil,
a sensor, operatively connected to the probe, functional to detect vibration in the soil to be tested at the site when the probe is in the sensing position in the soil, the sensor configured to provide an output indicative of sensed vibration, and
a processing unit, operatively connected to the sensor, functional to acquire data indicative of vibration sensed in the soil to be tested when the probe is in the sensing position in the soil, the processing unit configured to receive the output indicative of sensed vibration from the sensor and store data derived therefrom for processing to determine an indicator of soil health based on sensed vibration in the soil to be tested during a period of sensing in which the probe is in the sensing position in the soil.

2. The soil bioacoustic or ecoacoustic meter of claim 1, wherein the sensor comprises one of: a contact microphone, an accelerometer.

3. (canceled)

4. The soil bioacoustic or ecoacoustic meter of claim 1, wherein the probe is a rigid probe.

5. The soil bioacoustic or ecoacoustic meter of claim 1, wherein the processing unit is operatively connected to the sensor by one of:
a wired connection, a wireless connection.

6. (canceled)

7. The soil bioacoustic or ecoacoustic meter of claim 1, provided as a manually portable apparatus.

8. The soil bioacoustic or ecoacoustic meter of claim 1, wherein the processing unit comprises an enclosure, and the processing unit and probe are connected to form a single device.

9. The soil bioacoustic or ecoacoustic meter of claim 8, wherein the sensor is packaged with the processing unit.

10. The soil bioacoustic or ecoacoustic meter of claim 1, wherein the processing unit comprises an enclosure and the processing unit and probe are formed as separate devices that are connected by a wired or a wireless connection.

11. The soil bioacoustic or ecoacoustic meter of claim 10, wherein the sensor is packaged with the probe.

12. The soil bioacoustic or ecoacoustic meter of claim 8, wherein the enclosure is a waterproof housing.

13. The soil bioacoustic or ecoacoustic meter of claim 1, wherein the processing unit comprises at least:
a microprocessor,
a data storage device,
a communication port,
a battery power source, optionally comprising a rechargeable battery, and
a power on/off element.

14. (canceled)

15. The soil bioacoustic or ecoacoustic meter of claim 14, wherein the communication port allows both data and power transfer.

16. The soil bioacoustic or ecoacoustic meter of claim 13, wherein the processing unit further comprises at least one of:
a display unit,
an audio output device,
an illumination device,
a reading routine activation element for initiating the taking and recording of sensed vibration readings,
a timer device for tracking a period during which readings of sensed vibration are taken and recorded.

17. The soil bioacoustic or ecoacoustic meter of claim 13, further comprising a wireless communication module, optionally the wireless communication module is configured to allow communication with at least one of: a global navigation satellite system, a remote device.

18-19. (canceled)

20. The soil bioacoustic or ecoacoustic meter of claim 1, configured to record the frequency and amplitude of sensed vibration.

21. The soil bioacoustic or ecoacoustic meter of claim 1, the processing unit configured to perform analysis of data derived from the output indicative of sensed vibration to generate an indicator of soil health based on sensed vibration during a period of sensing in which the probe is in the sensing position in the soil.

22. The soil bioacoustic or ecoacoustic meter of claim 21, wherein the generated indicator of soil health based on sensed vibration during a period of sensing in which the probe is in the sensing position in the soil takes the form of a number on a proprietary scale.

23. The soil bioacoustic or ecoacoustic meter of claim 21, wherein said analysis comprises processing said data derived from the output indicative of sensed vibration with reference to pre-obtained data indicative of vibration sensed in soil.

24. (canceled)

25. A method of determining an indicator of soil health of a site based on biological activity of organisms that generate noise or vibrations, comprising:
(i) receiving the soil bioacoustic or ecoacoustic meter of claim 1,
(ii) inserting the penetrating tip of the probe into soil to be tested at the site to locate the probe in a sensing position in the soil, and
acquiring data indicative of vibration sensed in the soil to be tested during a period of sensing in which the probe is in the sensing position in the soil, and
(iii) processing the data acquired at (ii) to determine an indicator of soil health based on sensed vibration.

26. (canceled)

27. The method of claim 25, further comprising:
(iv) acquiring second data indicative of vibration sensed in the soil to be tested during a second period of sensing in which the probe is in the sensing position in the soil to be tested at the site, wherein the second period of sensing is at a later time than said period of sensing at (ii), and
(v) comparing the second data acquired at (iv) with the data acquired at (ii) to identify whether the recorded sound/vibration of the soil at the site is stable, increasing or decreasing
